(19) United States
(12) Patent Application Publication
(10) Pub. No.: US 2010/0042012 A1
(43) Pub. Date: Feb. 18, 2010

(54) Diagnostic device for remote sensing and transmitting biophysiological signals

(76) Inventor: Karim Alhussiny, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 12/192,607

(22) Filed: Aug. 15, 2008

(51) Int. Cl. A61B 5/04

(52) U.S. Cl. 600/546

(57) ABSTRACT

A diatrophic, bio-physiological interface is self-contained with onboard intensification, filtering, and signal processing and is wirelessly enabled (idio-electrode), with multiple sensory system for bio-physiological measurements, described herein utilizes spatially resolved potential profiles from a cluster of mini electrodes to form constituent sets comprising mini sensorial electrodes. The sets of sub electrodes containing the clusters are jointly optimized to attain measurable gradient of some diagnostic value. The present invention provides a distinct lead-free single electrode that is rotationally invariant with onboard Digital Signal Processor for arrhythmia detection, source encoding, and passive and active wireless transmission. Additionally, in one aspect of the present invention the lead-free idio-electrode bio-physiological adapter allows for utmost clinical operational freedom and dramatically obviates the needs for leads of any length that invariably encumber the acquisition and performance of electrocardiogram recordings of any sort.

### TECHNICAL FIELD

The present invention relates to a medical diagnostic device for monitoring biophysiological measurements and the transmission thereof. The present invention provides a self-contained macro-electrode with onboard amplifier, filter, and signal processor and wireless transmitter and more specifically the apparatus described herein utilizes spatially resolved potential profiles from a macro-electrode comprising cluster of sensorial sub-electrodes.

### BACKGROUND OF THE INVENTION

Interacting cardiac electric fields result in cardiac potentials that may be sensed through the body surface of a subject with metallic or gel electrodes. An electrode is essentially a transducer transforming the charges in electrolytes i.e., anions and cations into electrons and vise-versa for metals in electronic circuits. Anions and cations move with the aid of a perpetual sodium pump that energizes the cell and creates an electrochemical gradient in the intra and extra cellular spaces. This anion and cation movement in the intra and extra cellular spaces along with the conduction system of the myocardium allows for an action potential to travel. Conduction, displacement current flow or capacitive currents flow from one cell to another cell eventually activates and contracts the ventricle pump. Electric fields from the activated myocardium project from within the body outward where subtending electrodes acquire and record the bio-potential on the body surface.

For over a century, there have been recognized benefits of electrocardiogram recordings. However, the diagnostic benefits of electrocardiogram recordings are often left unrealized due to lack of full clinical exploitation. One possible reason for this lack of clinical exploitation may be due to relative difficulties in electrocardiogram administration. In general, electrocardiogram administration requires manipulating a bulky apparatus. Due to this inherent bulkiness, electrocardiogram administration is restricted mainly to clinics, hospitals, and emergency rooms. The efficiency in acquiring an electrocardiogram recording becomes more important when a critical event is timely detected and reported.

Perhaps complexities and perceived problems have been bolstered in association with affixing the recognized standard 12 leads to subjects. To properly utilize the standard 12 leads, subjects must be affixed with all 10 electrodes in the proper anatomical position. Affixing a subject with 12 leads may arguably be justified but not warranted in every case, and certainly should not be generalized such that it needlessly limits the benefit. The benefits garnered from a timely electrocardiogram transmission and interpretation potentially may lead to a subsequent preemption or subsequent intervention measure.

It can be expected that an easy to use macro-electrode will encourage widespread use. Such use is especially compelling when the macro-electrode incorporates such a wide range of technology and includes a foolproof protocol for affixation to a subject. For example, an easily affixed macro-electrode gives one the ability to monitor intervals of the cardiac cycle in real time. This real time monitoring is extremely beneficial in that intercepting cardiac events will enhance the overall healthcare. Additionally, early detection of otherwise undetected cases may lead to a reduction of the growing financial burden seen in the healthcare industry.

Currently, electrocardiogram recordings are widely used in clinical medical practice to detect electrical disturbances that are characteristic of cardiac abnormalities. However, the utility of such devices has several limitations. For example, most devices are bulky. These bulky devices relying on multiple electrodes joined by leads for acquisition. The standard example includes 12 leads that require 10 electrodes for acquisition. Even in the case of fewer electrodes, the acquisition devices requires leads connected separate electrodes. The necessary connectivity between the lead and the electrode remains a major and fundamental obstacle for realizing the full benefit from such devices.

Electrocardiogram recordings are based on measuring the potential difference from at least a pair of electrodes. These electrodes are distinctly separated and must be connected with leads that terminate in the amplified stage. A standard example includes 10 electrodes connected to 12 leads or the Frank set which is a three lead set in an orthogonal arrangement. In each case, the electrodes are connected with wires (leads) to the recording device.

The angle formed between the myocardium muscle fibers and the set of miniature electrodes influence the orientation and the grouping constituent clusters. With respect to the sequence of activation, the spread of the activation stimulus moves from endocrinal sites outward to the transmural space. This space is heavily affected by the anisotropic properties of the ventricular muscle. It is intuitive that excitation of the wavefront will spread more rapidly along the long axes of the cardiac cell than in the transverse direction. In ventricular walls, fibers are oriented roughly parallel to both endocardial and epicardial surfaces, however there are some transverse connections between cells. Therefore the spread from one endocardial point may be viewed as oblique. This means that there is a predominant axial movement along the length of the fiber with minimum movement perpendicular or transverse through the fiber. The cumulative effects of cardiac field results in variations in potential profiles.

To further complicate the situation, physiological and pathological variations across the human population also contribute to potential profile variations on body surface. It is common knowledge that a healthy heart may vary in its electrical axes. This is known as a normal variant. In some pathological cases, significant deviations exist such as myocardial hypertrophy. As a consequence to account for the significant variations that may exist across a population, the criterion in forming the sets and subsets of the sub-electrodes or miniature electrodes is that these sets are not necessarily adjacent. A maximum of three groups of electrode subsets form as the potential variations dictate the subset formation. Perhaps, the subsets form toward the direction of a maximum local sensed gradient from the cardiac area under study. Forming the subset of electrodes is somewhat like how sunflowers track the sun and how sunflowers align perpendicular to the sun.

As described above, the spatial potential spread and variations in the iso-potential lines continuum which may be spatially divergent or even highly restricted in certain areas on body surface influences the selection of the subset of electrodes. The intrinsic variation in cardiac potential maps across a population coupled with the difficulty in using biophysiological sensing technology drives the development of a diagnostic device comprising a cluster of sub-electrodes or miniature electrodes. It is understood that bipolar electrodes may diminish the contributions from remote potentials. However, bipolar measurements when confined to relatively small areas can accentuate and reveal contributions from remote potentials.

Several electrode arrangements namely, patches, have been proposed and described. However, for utmost ease of usage, the underpinning principal or challenge remains that the electrodes be adjacent, sufficiently and spatially separated. To avoid this fundamental necessity, others have demonstrated embedded wires in the lamination in several arrangements within patches. However, the obstacle remains that these electrodes are contained within a larger patch whose electrodes must be connected by wires with relatively large straddling separations. Furthermore, no prior art has shown a full acquisition of ECG with memory, full duplex transmitter and a receiver onboard or a single electrode with signal processing and a battery on area spanning less than the area of a typical electrode or an electrode autonomously contained on the same macro electrode of any shape limited to that area.

Nowhere does the prior art describe an iterative process for optimizing a diagnostic signal from a subset of electrodes contained within a cluster of electrodes. Additionally, the prior art does not describe how to obtain the necessary orientation of the sub-electrodes necessary to obtain the optimal biphysiological signal. There is a need to optimize a clinically diagnostic potential gradient from a single electrode comprising clusters of sub-electrodes within an one-inch by one-inch area.

Nowhere does the prior art describe formulating a system that delineates the logical steps needed to determine an algorithm that methodically identifies a structured approach to obtain a measurable bio-potential that is confined to a small area from a cluster of highly localized miniature electrodes. Secondly, the invention includes a design comprising a single small electrode. Ideally, this should be a miniature electrode that is autonomous, easily affixable, and contains a sensory system with detection and transmission capabilities. Thirdly, the prior art lacks an algorithm describing how to collectively measure, obtain, and optimize the end-to-end processes to achieve a diagnostic quality potential in a confined area. And fourthly, the prior art is deficient in demonstrating the specific intricacies of end to end design and ease of implementation to include the AFE, DSP and real time joint adaptive capabilities of hardware, method for transmissions, power supply, circuit components and within an area of a single typical electrode. And finally, the prior art is deficient in recognizing the value in measuring potential contained to highly localized area and the ability to relate the resulting constellation to measures of muscle and tissue deterioration.

The present invention fulfills this longstanding need and desire in the art.

### BRIEF SUMMARY OF THE INVENTION

The present invention provides a device and methods of sensing a biopyhsiological signal of diagnostic quality. In particular embodiments, the invention concerns acquiring a biophysiological signal from a subject using a wireless macro-electrode. In specific embodiments the invention is useful in detecting signals from skeletal muscle tissue, brain tissue, the eye, neurological tissue, nerve tissue, heart muscle, exposed brain tissue and epithelium tissue. In specific embodiments, the subject is a human.

In particular embodiments of the present invention, there is a macro-electrode device for remote sensing of a biophysiological signal comprises a substrate, said substrate comprising a plurality of sub-electrodes, said substrate forming one end of said macro-electrode, a power source, said power source is removably coupled to said substrate, and a processing unit, said processing unit removably coupled to said power source wherein said substrate, power source and processing unit form an integrated, unitary device. In specific embodiments, at least one sub-electrode is a receiver and at least one sub-electrode is an explorer. In other embodiments, at least one sub-electrode is a ground sub-electrode. In certain embodiments, each sub-electrode is connected to the power source. In some examples of the present invention, the power source is a battery. In additional embodiments, the battery is rechargeable.

In certain embodiments of the present invention, the power source has a power connection to the processing unit and a data transfer connection from each sub-electrode to the processing unit. In particular embodiments of the present invention, the processing unit comprises a means for acquiring data, a means for optimizing the biophysiological signal, a means for detecting an anomaly in the biophysiological signal, a means for transmitting the biophysiological signal, a means for storing data. In some embodiments, the processing unit further comprises means for transmitting and receiving speech.

In one embodiment of the present invention, at least two macro-electrode acquire a biophysiological signal and one macro-electrode is the master-electrode and the remaining macro-electrodes are slave-electrodes. In additional embodiments, the biophysiological signals acquired are synchronized and the slave-electrode transmits data to the master-electrode and the master-electrode transmits the synchronized signal. In particular embodiments, the biophysiological source is selected from the group consisting of skeletal muscle tissue, brain tissue, the eye, neurological tissue, nerve tissue, heart muscle, exposed brain tissue and epithelium tissue. In an alternate embodiment, the substrate comprises a circuit board containing an amplifier.

In one embodiment of the present invention, there is a method for remote sensing of a biophysiological signal with a macro-electrode comprising the steps of acquiring the biophysiological signal, filtering the biophysiological signal, selecting the permutation of sub-electrodes that optimizes the filtered biophysiological signal wherein the optimized signal results in a baseline signal, and wirelessly transmitting the baseline signal to a receiver. In some embodiments, the biophysiological signal is acquired from the group consisting of skeletal muscle tissue, brain tissue, the eye, neurological tissue, nerve tissue, heart muscle, exposed brain tissue and epithelium tissue. In specific embodiments, the biophysiological signal is acquired at a rate of per 1/300 second. In certain embodiments, the biophysiological signal is acquired between 0.5 Hz and 10,000 Hz. In some embodiments, the biophysiological signal is filtered to obtain a signal between 0.5 Hz to 10,000 Hz. In specific embodiments, the biophysiological signal is filtered to obtain a signal between 0.5 Hz to 60 Hz. In additional specific embodiments, the biophysiological signal is filtered to obtain a signal between 0.5 Hz to 50 Hz. In other embodiments, optimizing the biophysiological signal is achieved by minimizing the noise and maximizing the signal. In further specific embodiments, anomalies in the baseline signal are detected by interpreting the deviations from the pattern created by the baseline signal.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other methods for carrying out the same purpose of the present invention. It should be also realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood form the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIG. 1 shows the macro-electrode wherein 101 represents the substrate, 102 represents the processing unit and 103 represents the compartment for the battery.

FIG. 2 shows a side view of the macro-electrode wherein 201 represents a USB port, 202 represents the battery compartment, 203 is the substrate and 204 is the processing unit.

FIG. 3 shows a top view of the macro-electrode wherein 301 is the memory component, 302 shows the remaining power in the battery source, 303 is the liquid crystal display (LCD), 304 shows the optional voice transmitter and receiver, and 305 shows the on/off switch.

FIG. 4 shows a bottom view of the macro-electrode wherein 401 represents a sub-electrode, and 402 represents the substrate. The substrate also has a portion that contains adhesive, 403, for attachment to the body surface of a subject.

FIG. 5 shows an expanded view of the macro-electrode wherein 501 represents the processing unit, 502 represents a power connection between the power source and the processing unit, 503 represents the sub-electrode, 504 represents the substrate, 505 represents a connection between the sub-electrode and the power source, 506 represents a connection to transfer data and/or power between the power source and the processing unit, 507 shows the male portion of the male/female connection which used to physically secure the components to each other.

FIG. 6 shows a diagram of how a plurality of macro-electrodes may be used to give the full range of operability as a standard 10 electrode 12 lead system for measuring the cardiac potential of a subject. 601 represents a macro-electrode that functions as a slave and 602 represents a master electrode.

FIG. 7 shows a macro-electrode wherein 701 is a gateway, 702 is a processing unit, 703 is a power source and/or defibrillator, and 704 is a sub-electrode containing substrate.

FIG. 8 shows an expanded view of a macro-electrode wherein 801 is a processing unit, 802 is a male/female connection that provides both a means to secure the components of the macro-electrode to each other, as well as a means to transfer both data and power, and 803 is a sub-electrode that has a male/female type connection wherein the connection provides both a means to secure the substrate to the power source, as well as a means to transfer both data and power, and 804 is the substrate which contains a plurality of sub-electrodes.

### DETAILED DESCRIPTION OF THE INVENTION

### I. Definitions

As used herein, “a” or “an” means one or more than one.

As used herein, the term “constituent sets” refer to either the 2 set model or the 3 set model compromising the mini electrodes and constituting the an exploring set, a reference set, and a ground set.

As used herein, the term “model” refers to either the sets of 2 or 3 clusters.

As used herein, the term “critical session” refers to a phase wherein an event of significance has been detected, stored and needs to be transmitted or transmission is underway. It is possible that a sight waiting delay is imposed due to a wireless network delay etc.

As used herein, the term “IP” refers to internet protocol which is a protocol used for communicating data across a packet-switched internetwork using the TCP/IP suite of protocols. The Internet Protocol suite, TCP/IP, is the set of communications protocols used for the Internet and other similar networks. It is named from two of the most important protocols in it: the Transmission Control Protocol (TCP) and the Internet Protocol (IP).

As used herein, the term “RF” refers to radio frequency which is a frequency or rate of oscillation within the range of about 3 Hz to 300 GHz. This range corresponds to frequency of alternating current electrical signals used to produce and detect radio waves.

As used herein, the term “measurable” refers to a detectable potential of any diagnostic value from waveform excursions.

As used herein, the term “FIR filter” is a type of digital filter. The impulse response, the filter's response to a Kronecker delta input, is “finite” because it settles to zero in a finite number of sample intervals. The FIR filter as used herein is exemplary, other electronic filters may be used as well.

As used herein, the term “macro-electrode”, refers to a group of two or more sub-electrodes.

As used herein, the term “master-electrode”, refers to a macro-electrode that controls and regulates the activity of other macro-electrodes which are referred to as “slave-electrodes”.

As used herein the term “slave-electrode”, refers to macro-electrodes that are controlled and regulated by a master-electrode.

As used herein, the term “idio-” is a prefix that refers to personal, private, distinct and/or separate.

As used herein, the “common mode rejection ratio” of a differential amplifier (or other device) measures the tendency of the device to reject input signals common to both input leads. A high common mode rejection ratio is important in applications where the signal if interest is represented by a small voltage fluctuation superimposed on a (possibly large) voltage offset, or when relevant information is contained in the voltage difference between two signals.

As used herein, the term “gateway” describes a communications network or a network node equipped for interfacing with another network that uses different protocols. A gateway may contain devices such as protocol translators, impedance matching devices, rate converters, fault isolators, or signal translators as necessary to provide system interoperability. It also requires the establishment of mutually acceptable administrative procedures between both networks. A protocol translation/mapping gateway interconnects networks with different network protocol technologies by performing the required protocol conversions. In some cases, a macro-electrode is configured to perform the tasks of a gateway. In specific examples, a master-electrode is configured to perform the tasks of a gateway.

As used herein, the term “idio-electrode”, “mini-electrode” and “sub-electrode” may be used interchangebly. The term “constellation” as used herein describes the cooperation between two or more sub-electrodes.

As used herein, the term “CSD” refers to “circuit switched data” which is the orginal form of data transmission developed for the time division multiple access TDMA-based mobile phose systems like Global System for Moble Communications (GSM). CSD uses a single radio time slot to deliver 9.6 kbit/s data transmission to the GSM network ans switching subsystem where it could be connected through the equivalent of a normal modem to the Public Switched Telephone Network (PSTN) allowing direct calls to any dial-up service.

As used herein, the term “SMS” refers to “short message service” which is a communications protocol allowing the interchange of short text messages between mobile telephone devices. Since its inception SMS has expanded from the transmission of text messages to include a number of other types of broadcast messaging.

### II. [The Apparatus]

Patches and electrode clusters for clinical electrocardiography have been described in the prior art. However, the prior art does not describe the three principals necessary for sensing a bio-potential signal. These principles include sensitivity, spatial resolution, and orientation of sub-electrodes wherein the sub-electrodes are located within clusters confined to a relatively small zone or a macro-electrode. Several principals are required to develop a macro-electrode for acquiring a biophysiological signal. First, a sensorial array is required to spatially resolve highly localized gradient profiles from a cluster of mini electrodes to form constituent sets comprising sub-electrodes under a specific decision rule. These clusters will provide at least two constituent sets from various members of the cluster to discern a measurable potential difference. Secondly, a procedure that minimizes the noise and maximizes the signal allows for bio-potential sensory acquisition through a virtual digital steering, selecting, grouping, and hence recording and monitoring from permutations of a plurality of small electrodes or sub-electrodes confined to the size of the typical ECG electrode. Thirdly, potential contributions from all possible permutations of the cluster of sub electrodes are combined and parsed into two or three macro constituent sets. Fourthly, the rotational invariance property is achieved by virtually steering of the miniature electrodes within cluster to obtain measurable potential difference. Another principal needed for developing a macro-electrode is a battery structure that provides power to the body of the electrode, its sensorial mini-electrodes and the onboard processing device. A wireless topology network model is also essential where a remote monitor can interrogate the portable idio-electrode. This combination of distinct principals and essential elements provides a distinct single electrode that is autonomous and lead-free which comprises an onboard DSP with arrhythmia detection capabilities, source encoding, and passive and active wireless transmission. In addition to detection capabilities, the macro-electrode is functional in a Holter mode.

The idio-electrode method partially combines elements of (1) bio-potential interface with a mini-sensorial cluster system to obtain a gradient from a highly localized potential; (2) a procedure that minimizes the noise and maximizes the signal obtained from two or more electrodes to form mini-electrodes constellation(s). These constellations are formed to at least attain a measurable divergence of cardiac field force vector with respect to a source vector, for example: additional elements include, (3) a sample of a subjects electrophysiological activity for arrhythmia monitoring and tracking and (4) a wireless network methodology to maintain connectivity and aid interrogation during critical sessions.

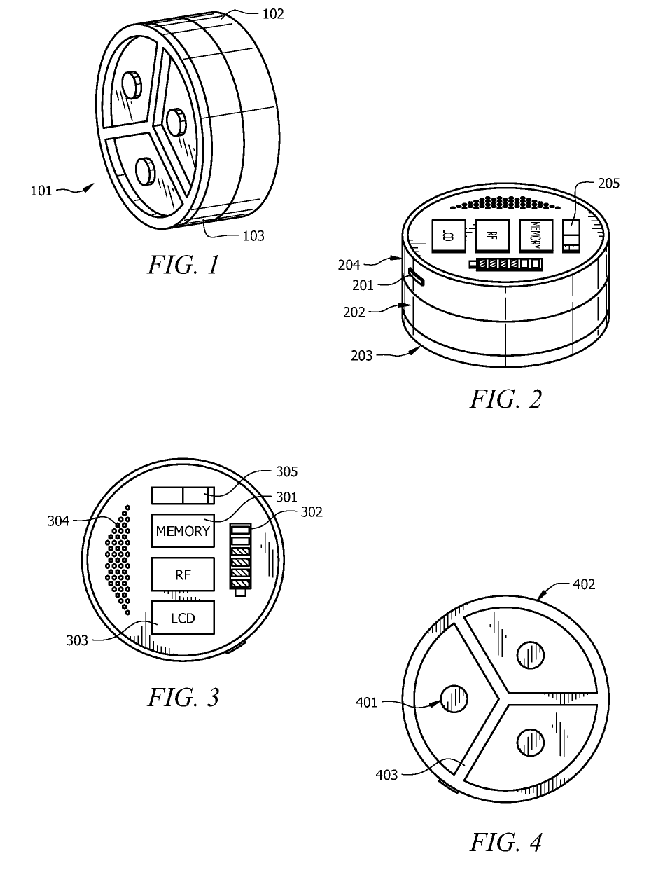

FIG. 1 shows a macro-electrode wherein 101 represents the substrate, 102 represents the processing unit and 103 represents the power source. The sub-electrodes are contained with the substrate 101. The substrate contains at least two sub-electrodes. The substrate 101 is the portion of the macro-electrode that comes in contact with the body surface of the subject. The substrate is affixed to the subject using an adhesive. In some examples, the adhesive covers the entire surface of the substrate excluding the surface of the electrodes. The substrate 101, the power source 103 and the processing unit 102 are removably coupled to each other through male/female type connections. FIG. 2 shows a side view of the macro-electrode wherein 201 represents a USB port, 202 represents the power source, 203 represents the substrate, and 204 represents the processing unit. The USB port 201 maybe used for both data transfer and as a port to charge the battery. The power source 202 is a compartment that has been customized to fit a battery or a power cell. In FIG. 2, the substrate 203 contains a plurality of sub-electrodes. The substrate 203 is the portion of the macro-electrode that comes in contact with the subject. The macro-electrode may be affixed to the subject in a number of ways. The preferred method to affix the macro-electrode to the subject is by adhesively coupling the substrate 203 to the subject. The processing unit 204 in addition to having a USB port 201 has an on/off switch 205. This on/off switch 205 gives an additional method to conserve energy.

FIG. 3 shows a top view of the macro-electrode wherein 301 shows the available memory in the onboard memory, 302 shows the remaining power in the battery or power cell, 303 is the LCD, liquid crystal display, 304 is the voice transmitter and receiver, and 305 is the on/off switch. In some examples, the LCD shows the status of the macro-electrode. This status may be slave for slave-electrode, master for master-electrode, passive or active, synchronized or unsynchronized. FIG. 4 shows a bottom view of the macro-electrode wherein 401 is the sub-electrode. FIG. 4 shows three sub-electrodes however there may be more. The number of sub-electrodes is limited by the available space on the substrate 402. In FIG. 4, 403 represents the portion of the substrate that is coated with adhesive. In FIG. 4, 403 is the portion of the substrate that affixes to the subject.

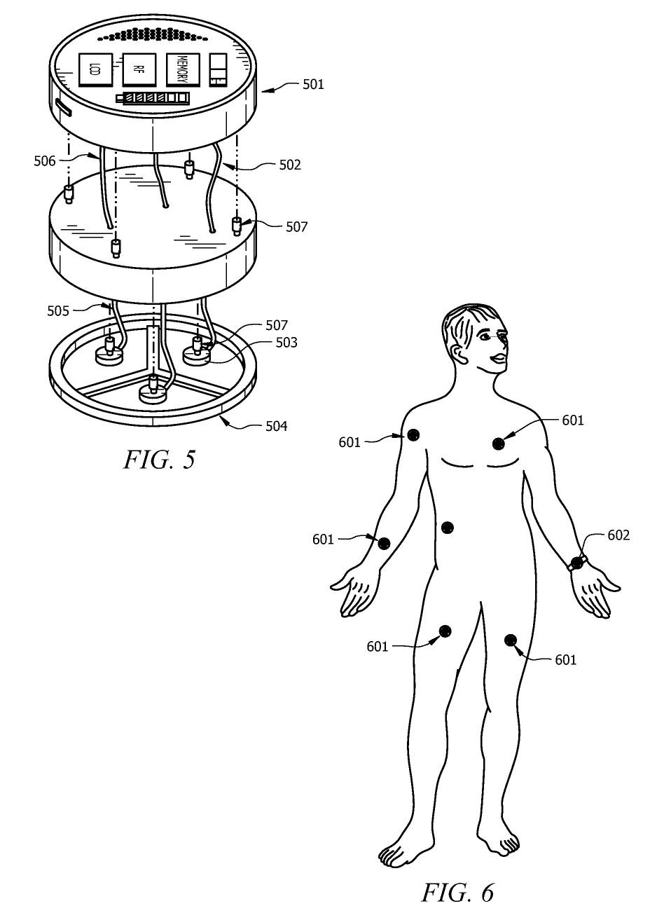

FIG. 5 is an expanded view of the macro-electrode, wherein 501 is the processing unit, 502 and/or 506 shows the connection between the power source and the processing unit. 502 and 506 may be used to transfer power and data. In some examples, 502 will sole transfer power and 506 will solely transfer data. In other examples, 506 will solely transfer power and 502 will solely transfer data. In additional examples, both 502 and 506 will transfer both power and data. In FIG. 5, 504 is the substrate which contains 503 the sub-electrode. In some examples the sub-electrode 503 is connected to the power source through wires 505. In alternate embodiments the sub-electrode 503 is connected to the power source through male/female type connections. 507 is the male/female connection that secures the substrate to the power source and secures the power source to the processing unit.

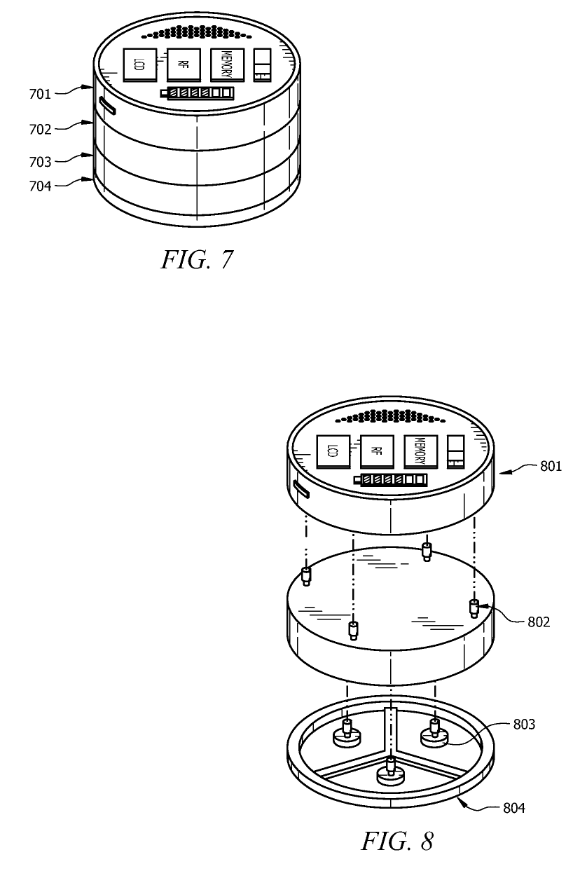

FIG. 7 shows an alternate embodiment of the present invention, wherein 701 is a gateway, 702 is a processing unit, 703 is a power source and/or a defibrillator, and 704 is the sub-electrode containing substrate. In this example, the macro-electrode may possess any number of functionalities. The gateway 701 may be comprised of a on/off switch, at least one cellular module, a high capacity battery, a RF transmitter/receiver, memory, processing capabilities, and/or a component to transmit and receive voice. The processing unit 702 may operate in active or passive modes. When the processing unit is in passive mode the electrodes are not being used to acquire a biophysiological signal and when the processing unit is in active mode the electrodes are being used to acquire a biophysiological signal. The macro-electrode may be place in active or passive mode by a user sending a signal to the gateway from a remote location.

Also as shown in FIG. 7, the power source 703 may be used as a defibrillator. The power source may send electrical pulses through the electrodes to the subject. These electrical pulses can be used to mimic the sequential activation of the heart. Also there is a reduction in myocardium stunning because a smaller amount of voltage is required in comparison to the standard defibrillator. Therefore, when the macro-electrode is being used to monitor the cardiac cycle, a remote user monitoring the subject may be able to manually stimulate the cardiac cycle until help arrives.

In some examples of the present invention, the components of the macro-electrode are removable. In this example, the components of the macro-electrode are connected through male/female type connections. Additionally, these male/female type connections may serve as connections to transfer and receive data and power to each component. FIG. 8 shows a macro-electrode wherein 801 is the processing unit, 802 is the male/female connection that connects the processing unit to the power source, 804 is the substrate that contains a plurality of sub-electrodes 803. For example in FIG. 8, the sub-electrodes 803 have a male type connection and the power source has its female counterpart. In this example, the male/female connection between the sub-electrode and the power source acts as both a means to secure the substrate to the power source and as a means to transfer power and data. These male/female type connections also may be used to secure the power source to the processing unit as well as transfer power and data from the power source to the processing unit. In certain situations, these male/female type connections are advantageous. For example, if the battery in the power source is low, then the battery can easily be replaced with a fully charged battery. Also, the male/female type connection allows for a three component macro-electrode as seen in FIG. 8 to be converted to a four component macro-electrode as seen in FIG. 7. Ultimately as technology improves, additional feature may be added to an existing macro-electrode by simply adding, replacing or removing a component of the macro-electrode.

### A. Acquisition of Bio-Potential Sensory Measurements

The sensory element of the present invention provides for the acquisition of a biophysiological signal such as the cardiac potential. Other measureable biophysiological sources include but are not limited to signals acquired from skeletal muscle tissue, brain tissue, contact lens electrode, neurological sources, nerve tissue, heart muscle, retina, exposed brain surface, and/or sleep apnea. The biophysiological activity is acquired through bipolar measurement which is the measure of the difference between two electrodes. These electrodes are referred to as the reference and the explorer. A third electrode referred to as the ground is used to measure the common mode rejection ratio. This third electrode eliminates the signals below 60 Hz that are common to the reference and explorer electrodes. The electrodes are grouped to form a cluster of electrodes. At least three electrodes are needed to form a cluster. The resulting cluster of sub-electrodes or miniature electrodes form the sensory element of the apparatus. In some situations, not all of the electrodes contribute to the biophysiological signal.

In some situations, the present invention utilizes temporal and spatial-resolved detection of bio-physiological potential to obtain discernable waveforms for diagnostic purposes. The resulting waveform is obtained from highly localized clusters of single or multiple electrodes from a body surface or from organs. In some cases, a minimum of one single electrode may be used to detect the cardiac electrical disturbance. Drawing upon and recognizing the fundamentals of electrochemical processes, inferences can be made with a great degree of certainty in discerning the spatial and temporal gradient divergence based upon the resulting spectra. Selection and the formation of the constellations during the cardiac cycle is dictated by those-electrodes coincident and subjacent to iso-potential contours of high divergence contributing the most to a measurable differential waveform.

The ground electrode in bipolar measurements can only introduce an inconsequential bias in highly localized bio-potential measurements. By correlating and analyzing the spectra of potential contours, a user may determine the placement of the electrodes as well as the minimum number of macro-electrodes needed to attain the desired signal. It is therefore conceivable that fewer than the 10 standard electrodes may be used to predict subjacent lesions or injuries.

The fundamental requirement of attaining a discernable gradient from a single electrode comprising clusters mini-electrodes, in a highly localized potential requires flexible and robust adjustment. Iso-potential lines or contours dictate that a specific orientation of the sub electrodes. It is not necessary for the sub electrodes to be adjacent or uniform. Additionally, not all of the sub-electrodes or miniature electrodes are required to detect the potential variability during the cardiac cycle. In addition, joint adaptive capabilities are required at the circuit level. The sub electrodes connect to a differential amplifier and the amplifier is powered by a battery on board the macro-electrode. The joint adaptive capabilities include but are not limited to capacitors, resistors as well as digital processing abilities.

B. Selecting the Permutation of Sub-Electrodes that Provides the Maximum Potential Gradient

All sub electrodes terminate into an addressable multiplexer and are controlled by instructions from a microprocessor, digital signal processor (DSP), or any other digital processor. Various miniature electrodes or sub-electrodes are combined into their prospective sets to form the minimum 2 or 3 constituent sets. These sets represent the potential points to obtain spatio-temporal waveform excursions, reflective of the cardiac generator that is least noisy. The sets of clusters, comprising the sub-electrodes, are arranged to discern or maximize the signal gradient with the least interference noise.

The selection of the two or three sub-electrodes within the electrode cluster may not necessarily form a adjacent set of sub-electrodes. This condition provides a resource for optimizing the maximum potential gradient. In some situations, the optimal mode is to combine miniature electrodes or sub-electrodes to contribute to a stable gradient preferably of some visually desired display that is free or indiscernibly tolerable, for example AC, alternating current, interference. On board the macro-electrode module is a set of amplifiers along with the addressable multiplexer which communicates all potential permutations of the sub-electrodes to a signal processing unit on board the macro-electrode unit. A set of amplifiers may be used to accommodate the selection of candidate clusters comprising the localized sub-electrodes. The macro-electrode can also be used as a hub to other electrodes forming a single or perhaps any standard electrode arrangement such as the standard 12 leads and/or the Frank set. In this situation one macro-electrode functions as the master-electrode and the remaining macro-electrodes function as slave-electrodes. The orientation selection process from sub-electrodes may be iterative until a desired bipolar potential is attained. The orientation selection process is performed over all possible subsets of the sub-electrodes of the cluster in the 1″×1″ area or less. In some cases, the orientation selection process may occur within the size of single electrode or within commercially available electrodes used in ECG recording.

All of the sub-electrodes will be parsed and optimized according to set of criteria to form sets of either two or three electrodes. The resulting set of electrodes may or may not be adjacent. In the situation where there are three electrodes in a set, the set comprises a explorer, a reference, and a ground electrode. In the situation where the desired biophysiological signal results in a ECG, the criterion is maximum ECG excursions falling in the ECG band and void of AC interference. In certain examples, the ground electrode may not be necessary. In this situation, two electrode form a set and each set contains at least one miniature electrode wherein one miniature electrode is sufficient for obtaining a diagnostic signal.

The two sub-electrodes that give the minimum noise and maximum signal provide one criterion for selecting the sub-set of electrodes. While it is desirable, this arrangement is not critical insofar as a sufficiently discernable measurement of any diagnostic value is obtained. The process in selecting the two sub-electrodes begins first by obtaining a differentially sampled electrical gradient. In some cases, the sampling may be digital. It is understood by those of skill in the art that other sampling methods may be used and are within the scope and spirit of the present invention. After sampling, the electrical gradient for every possible permutation of sub-electrode by sub-electrode is computed. The sub-electrode by sub-electrode that gives the minimum noise and the maximum signal is selected. In some examples of the present invention, all remaining sub-electrodes may be used to return current such so that the diagnostic measurements are obtained over the electrode reference with least interference from AC power lines and/or any other interfering source. In some examples of the present invention, at least one sub-electrode may be used to return current. In some situations, the third reference is not needed. In other examples, a FIR notch filter is applied to reduce the AC magnification. The signal may be magnified following the notch at 60 or 50 Hz.

In some examples of the present invention the macro-electrode acquires a biophysiological signal between 0.25 Hz and 10,000 Hz, 0.25 Hz and 9,500 Hz, 0.25 Hz and 9,000 Hz, 0.25 Hz and 8,750 Hz, 0.25 Hz and 8,500 Hz, 0.25 Hz and 8,000 Hz, 0.25 Hz and 7,750 Hz, 0.25 Hz and 7,500 Hz, 0.25 Hz and 7,250 Hz, 0.25 Hz and 7,000 Hz, 0.25 Hz and 6,750 Hz, 0.25 Hz and 6,500 Hz, 0.25 Hz and 6,250 Hz, 0.25 Hz and 6,000 Hz, 0.25 Hz and 5,750 Hz, 0.25 Hz and 5,500 Hz, 0.25 Hz and 5,250 Hz, 0.25 Hz and 5,000 Hz, 0.25 Hz and 4,750 Hz, 0.25 Hz and 4,500 Hz, 0.25 Hz and 4,250 Hz, 0.25 Hz and 4,000 Hz, 0.25 Hz and 3,750 Hz, 0.25 Hz and 3,500 Hz, 0.25 Hz and 3,250 Hz, 0.25 Hz and 3,000 Hz, 0.25 Hz and 2,750 Hz, 0.25 Hz and 2,500 Hz, 0.25 Hz and 2,250 Hz, 0.25 Hz and 2,000 Hz, 0.25 Hz and 1,750 Hz, 0.25 Hz and 1,500 Hz, 0.25 Hz and 1,250 Hz, 0.25 Hz and 1,000 Hz, 0.25 Hz and 750 Hz, 0.25 Hz and 500 Hz, 0.25 Hz and 250 Hz, 0.25 Hz and 100 Hz, 0.25 Hz and 75 Hz, 0.25 Hz and 50 Hz, 0.25 Hz and 25 Hz, 0.25 Hz and 10 Hz, 0.25 Hz and 10,000 Hz,25 Hz and 10,000 Hz, 50 Hz and 10,000 Hz, 60 Hz and 10,000 Hz, 75 Hz and 10,000 Hz, 100 Hz and 10,000 Hz, 150 Hz and 10,000 Hz, 200 Hz and 10,000 Hz, 225 Hz and 10,000 Hz, 250 Hz and 10,000 Hz, 275 Hz and 10,000 Hz, 300 Hz and 10,000 Hz, 325 Hz and 10,000 Hz, 350 Hz and 10,000 Hz, 375 Hz and 10,000 Hz, 400 Hz and 10,000 Hz, 425 Hz and 10,000 Hz, 450 Hz and 10,000 Hz, 475 Hz and 10,000 Hz, 500 Hz and 10,000 Hz, 525 Hz and 10,000 Hz, 550 Hz and 10,000 Hz, 575 Hz and 10,000 Hz, 600 Hz and 10,000 Hz, 625 Hz and 10,000 Hz, 650 Hz and 10,000 Hz, 675 Hz and 10,000 Hz, 700 Hz and 10,000 Hz, 725 Hz and 10,000 Hz, 750 Hz and 10,000 Hz, 775 Hz and 10,000 Hz, 1000 Hz and 10,000 Hz, 2000 Hz and 10,000 Hz, 3000 Hz and 10,000 Hz, 4000 Hz and 10,000 Hz, 5000 Hz and 10,000 Hz, 6000 Hz and 10,000 Hz, 7000 Hz and 10,000 Hz, 8000 Hz and 10,000 Hz, 9000 Hz and 10,000 Hz, 2.25 Hz and 100 Hz, 10 Hz and 90 Hz, 20 Hz and 80 Hz, 30 Hz and 70 Hz, 40 Hz and 60 Hz, 50 Hz and 60 Hz, 35 Hz and 75 Hz, 45 Hz and 65 Hz, and/or any combination thereof.

In some examples of the present invention the macro-electrode filters the acquired biophysiological to obtain a signal between 0.25 Hz and 10,000 Hz, 0.25 Hz and 9,500 Hz, 0.25 Hz and 9,000 Hz, 0.25 Hz and 8,750 Hz, 0.25 Hz and 8,500 Hz, 0.25 Hz and 8,000 Hz, 0.25 Hz and 7,750 Hz, 0.25 Hz and 7,500 Hz, 0.25 Hz and 7,250 Hz, 0.25 Hz and 7,000 Hz, 0.25 Hz and 6,750 Hz, 0.25 Hz and 6,500 Hz, 0.25 Hz and 6,250 Hz, 0.25 Hz and 6,000 Hz, 0.25 Hz and 5,750 Hz, 0.25 Hz and 5,500 Hz, 0.25 Hz and 5,250 Hz, 0.25 Hz and 5,000 Hz, 0.25 Hz and 4,750 Hz, 0.25 Hz and 4,500 Hz, 0.25 Hz and 4,250 Hz, 0.25 Hz and 4,000 Hz, 0.25 Hz and 3,750 Hz, 0.25 Hz and 3,500 Hz, 0.25 Hz and 3,250 Hz, 0.25 Hz and 3,000 Hz, 0.25 Hz and 2,750 Hz, 0.25 Hz and 2,500 Hz, 0.25 Hz and 2,250 Hz, 0.25 Hz and 2,000 Hz, 0.25 Hz and 1,750 Hz, 0.25 Hz and 1,500 Hz, 0.25 Hz and 1,250 Hz, 0.25 Hz and 1,000 Hz, 0.25 Hz and 750 Hz, 0.25 Hz and 500 Hz, 0.25 Hz and 250 Hz, 0.25 Hz and 100 Hz, 0.25 Hz and 75 Hz, 0.25 Hz and 50 Hz, 0.25 Hz and 25 Hz, 0.25 Hz and 10 Hz, 0.25 Hz and 10,000 Hz,25 Hz and 10,000 Hz, 50 Hz and 10,000 Hz, 60 Hz and 10,000 Hz, 75 Hz and 10,000 Hz, 100 Hz and 10,000 Hz, 150 Hz and 10,000 Hz, 200 Hz and 10,000 Hz, 225 Hz and 10,000 Hz, 250 Hz and 10,000 Hz, 275 Hz and 10,000 Hz, 300 Hz and 10,000 Hz, 325 Hz and 10,000 Hz, 350 Hz and 10,000 Hz, 375 Hz and 10,000 Hz, 400 Hz and 10,000 Hz, 425 Hz and 10,000 Hz, 450 Hz and 10,000 Hz, 475 Hz and 10,000 Hz, 500 Hz and 10,000 Hz, 525 Hz and 10,000 Hz, 550 Hz and 10,000 Hz, 575 Hz and 10,000 Hz, 600 Hz and 10,000 Hz, 625 Hz and 10,000 Hz, 650 Hz and 10,000 Hz, 675 Hz and 10,000 Hz, 700 Hz and 10,000 Hz, 725 Hz and 10,000 Hz, 750 Hz and 10,000 Hz, 775 Hz and 10,000 Hz, 1000 Hz and 10,000 Hz, 2000 Hz and 10,000 Hz, 3000 Hz and 10,000 Hz, 4000 Hz and 10,000 Hz, 5000 Hz and 10,000 Hz, 6000 Hz and 10,000 Hz, 7000 Hz and 10,000 Hz, 8000 Hz and 10,000 Hz, 9000 Hz and 10,000 Hz, 2.25 Hz and 100 Hz, 10 Hz and 90 Hz, 20 Hz and 80 Hz, 30 Hz and 70 Hz, 40 Hz and 60 Hz, 50 Hz and 60 Hz, 35 Hz and 75 Hz, 45 Hz and 65 Hz, and/or any combination thereof.

In some cases, local potentials are resolved by their sum and difference measurements. Therefore, any permutations of standard ECG recordings can be identified as Lead I, II, II, AVR, V1-V6 or any other known recordings and any linear combination to form standard or derived potential measurements.

At each macro-electrode, there are several stages of amplification with a proper gain to accommodate any classical arrangement such as the standard 12 leads, Frank set and/or any other derived cluster used for monitoring.

### C. Detecting Anomalies in the Baseline Signal

In one method of detecting an anomaly in the baseline signal, arrhythmia detection is used. It should be understood by one skilled in the art, that arrhythmia detection is exemplary and that other examples may fall within the scope of the present invention without deviating from the nature and spirit of the present invention. The algorithm for arrhythmia detection is preferably specific for any individual, relying on patient base line wherein a remote operator marks fiducial points automatically. The automatic or “blind mode” of deciding based upon the constituent sets of fiducial points relies on detecting which is initially done by trending, and convergence which is achieved through stepped correlation of matching, classifying and validating the resulting complex. This algorithm assumes pseudo-repeatable, relative regularity, convergent or semi-periodic complexes. In the case when the complex fails to converge, the event signals a potential emergency since convergence of some sort is highly likely with repeatable and successive complexes. The lack of convergence over the preliminary phase of training and acquisition may indicate chaotic electrical activity in the heart. This is also indicative of multi-morphic complexes, which, in relative terms, should not be the case under non-emergency situation.

In the case of supervised or directed provisioning by a remote operator, a paramedic or a nurse, these fiducial points are picked interactively from for example, a display screen and inserted as part of the algorithm firmware. Intrinsic excursion from incoming data is compared to the baseline. If an alternating rhythm is present such as the case when a patient reverts from atrial fibrillation and back to normal and so on, the algorithm will store both rhythm as admissible base lines. In this common situation both rhythms are deemed admissible, albeit different, but do not warrant an emergency. It is not uncommon for a subject to have multiple variations in cardiac rhythms. In this situation, when an additional rhythm is deemed admissible, the algorithm will allow the macro-electrode to store this rhythm as a baseline. It is not likely that a subject will have a plurality of admissible irregular rhythms and even more unlikely that these irregular rhythms will not warrant a medical emergency. The algorithm will maintain and report increases in admissible baseline switching and the rate from one base line to another.

### D. Transmission

One aspect of the present invention is the network; namely, the remote receiver and the networking segment that maintains connectivity and enable robust wireless communications through interrogation of the portable electrode. The reduction of the data and elastic signaling during critical sessions enables maintenance of connectivity between the mobile patient and the cellular tower or other communication point. A mobile subject may be interrogated by any of the two prevailing modes such as legacy of circuit switching CSD or the currently emerging packet switching over IP. In the case of CSD, the remote patient's data may be analyzed by using a number assigned to a mobile phone. The case for data over IP is somehow slightly different in that a mobile patient is not seen and is only seen by a local network cluster, as an IP entity over the internet backbone if the remote interrogator sends an SMS burst to activate him/her. By using SMS from a server to reach a remote patient, a full duplex session is established where the mobile patient is a wakened to start transmitting upon the SMS interrogation command. It should be noted that a remote mobile IP user, albeit connected to the network, is “out of sight” in the sense that he/she can not be seen as an autonomously addressable IP entity except by a local router.

Power conservation is a central issue for the viability of the mobile single electrode The data source encoder on board the macro-electrode extracts duplications inherent in the biophysiological waveform. The processor builds correlation functions that measure the “degree of sameness”, within the raw waveform, to extract and “concentrate” representative signal. This representative signal may now be stored or transmitted. Power may be conserved in this “concentration” process through concentrated bits. At the receiver decision space, the decision of whether a bit is one or zero is the amount of energy associated with received bit. The measured effective energy in the decision space is more important than the instantaneous power that is generally required for high data rates. As noted the energy of a contended bit more important in the decision space. Energy is the product of power and time (E=PT). Longer bits resulting from lower data rates (T=1/R, where R is data rate in bits per seconds), can have more energy simply because longer bits remain in the decision space longer which gives the receiver ample time to make a decision. Therefore it can be ascertained that longer bits will consume more energy without increasing the power. This rationale allows for the reduction of the power requirements. The effective power reduction is achieved by “well concentrating”, i.e., source coding, the representative bits and casts each over a longer time period through the RF link or Infrared. An alternative method of preserving power is by providing a the macro-electrode that needs only to be in close vicinity of a local receiving and more powerful gateway. In this case the electrode may or may not have a operational cellular module and the gateway module may optionally provide wireless access to a circuit switched network or an opening to the internet.

### E. Power Source

The macro-electrode may be powered through an external power source or through an internal power source on board the macro-electrode. In the situation where the macro-electrode is powered using an external power source, the external power source may be a battery, a solar cell, an electrical outlet or a combination thereof. In the case, where an electrical outlet is used to power the macro-electrode, power may be received through a AC or a DC, direct current, source. Some typical examples of an AC or DC source may include the standard household outlet or an outlet commonly found in a vehicle. The power source may contain any standard type of connection for receiving power such as a USB, universal serial bus, port. The power source is connected to the processing unit, substrate and/or electrodes through any variety of common male/female type connections.

In some cases, the power source is a battery onboard the macro-electrode. In this situation the macro-electrode contains a customized compartment for housing the desired battery type. This compartment is customized with leads, direct connections, or any variation of connections for supplying power to both the processing unit and to the electrodes. The batteries that may be using in the present invention include but are not limited to SR521, AGO, 379, SR41, AG3, LR41, D384/392, LR41 (alkaline), SR41 (silver oxide), 1135SO, silver oxide), 1134SO (silver oxide), 32 (alkaline), 42 (silver oxide), 1.50 (alkaline), 1.55 (silver oxide), SR43, AG12, LR43, D301/386, LR43 (alkaline), SR43 (silver oxide), 1133SO (silver oxide) 1132SO (silver oxide), 80 (alkaline), 120 (silver oxide), 1.50 (alkaline), 1.55 (silver oxide), SR44, AG13, LR44, D303/357, LR44 (alkaline), SR44 (silver oxide), 1166A (alkaline), 1107SO (silver oxide), 1131SOP (silver oxide), 150 (alkaline), 200 (silver oxide), 1.50 (alkaline), 1.55 (silver oxide), SR48, AG5, D309/393, SR48 (silver oxide), 1136SO (silver oxide), 1137SO (silver oxide), 70 (silver oxide), 1.55 (silver oxide), SR54, AG10, LR54, 387S/D389/390, LR54 (alkaline), SR54 (silver oxide), 1138SO (silver oxide), 100 (alkaline), 70 (silver oxide), 1.50 (alkaline), 1.55 (silver oxide), SR55, AG8, D381/391, SR55 (silver oxide), 1160SO (silver oxide), 40 (silver oxide), 1.55 (silver oxide), SR57, SR927W, AG7, D395/399, LR57 (alkaline), SR57 (silver oxide), 116550 (silver oxide), 55 (silver oxide), 1.55 (silver oxide), SR58, AG11, D361/362, SR58 (silver oxide), 1158SO (silver oxide), 24 (silver oxide), 1.55 (silver oxide), SR59, AG2, D396/397, SR59 (silver oxide), 1163SO (silver oxide), 30 (silver oxide), 1.55 (silver oxide), SR60, AG1, D364, SR60 (silver oxide), 1175SO (silver oxide), 20 (silver oxide), 1.55 (silver oxide), SR66, AG4, D377, SR626SW, SR66 (silver oxide), 1176SO (silver oxide), 26 (silver oxide), 1.55 (silver oxide), SR69, AG6, R371, SR69 (silver oxide) or a combination thereof.

In some examples, the electrodes are connected to a circuit board containing an amplifier. In this situation the circuit board is connected to the power source through wires. This arrangement is ideal when there is a fair amount of rotational movement in the electrodes. When the subject moves it causes the macro-electrode to move and the movement of the macro-electrode (battery) lead to rotational movement of the electrodes that cause gaps between the electrode and the body surface of the subject. These gaps result in fluctuations in the half cell potential which lead to distorted signals.

### F. Processing Unit

The macro-electrode also comprises a processing unit. This processing unit has a number of functions including, but not limited to, processing and filtering the biophysiological signal, finding the permutation of sub-electrodes that minimize the noise and maximize the signal, wirelessly transmitting and receive data, synchronizing the signal acquired from additional macro-electrodes where necessary and/or storing data. The processing unit is removably coupled to the power source through any common male/female type connection. The processing unit is electrically coupled to the battery and the substrate. The electrical connections may be of any type commonly used in the art to transfer power and/or data.

The processing unit may also transmit and receive speech through the cellular communications module used to transmit the biophysiological signal. This feature allows a subject to indentify symptoms by voice. This voice message is time stamped which allows a doctor or nurse to correlate the voice message with a specific moment in the biophysiological data graph. Such processing units are known to those of skill in the art.

### G. Multiple Master-Electrodes

The following example is included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

In some situations, multiple macro-electrodes may be used to acquire, process and transmit a biophysiological signal. Each macro-electrode is equipped with a RF transreceiver which may communicate individually (single or plurality) in synchronized or unsynchronized mode. In some examples, the macro-electrode communicates to a gateway. This communication may be cellular based or based upon any RF modality. Examples of RF modalities include but are not limited to WiMAX, ZigBee, Bluetooth and Wi-Fi. In some examples, at least two macro-electrodes are preset wherein one macro-electrode is a master-electrode and the remaining macro-electrodes are slave-electrodes. The individual macro-electrodes are further synchronized to multiplex all channels at the receiver. In some cases, the receiver is the master-electrode. The receiver is the gateway that contains the cell for any RF transmitter receiver. The receiver also may be worn as a watch, necklace, article of clothing or in a holster.

For example, in measuring the EKG, the master-electrode may be worn as a watch. Each macro-electrode in this case would measure sub-adjacent potential from the local electrode wherein the subadjacent potential is sampled and transmitted to the gateway. In this example the gateway is a watch. However, the gateway may also be a necklace, holster, etc.

In another example of the present invention, the macro-electrodes are used to measure ST-segment. The ST-segment is usually measured with 12 leads. In this example, the macro-electrodes are in constellation just as the with the standard case which uses 12 leads (10 electrodes). It is understood by one skilled in the art that other arrangements are possible. Each sub-adjacent cardiac potential is sampled and transformed into RF bytes that are transmitted to the remote gateway that is portable such as a watch or necklace. These electrodes must be synchronized to one clock for their combination to be meaningful. Although each potential obtained can certainly be used alone for monitoring purposes for arrhythmia but may have (individually) limited ST diagnostic value since the subadjacent potential reveal the same portion of the myocardium (local) and injury currents may well be suppressed at best or vanish completely. Averaging of localized potential is representative of local potential such as right arm or any other standard electrode position. These local potentials are combined by transmitting them to another processing unit, such as a master electrode. This master-electrode can be worn as a watch wherein the multiplexed RF links from various slave-electrodes are synchronized in time so that a meaningful signal is obtained.

FIG. 6 shows how one may use multiple macro-electrodes to obtain similar diagnostic information as obtained through the standard 10 electrode 12 lead setup. In FIG. 6, 601 shows a slave-electrode and 602 shows a master-electrode. The arrangement in FIG. 6 is exemplary and it is understood by those of skill in the art that the arrangement may be modified to obtain the desired diagnostic information.

In situations wherein more than one macro-electrode is used, one electrode is a master-electrode and the signals of remaining slave-electrodes are synchronized and transmitted to the master-electrode. Echoing is the basis for synchronization. In the synchronization mode the slave-electrodes are initially in a listening mode. The slave-electrodes receive a burst of data packets from the master-electrode. This burst of data packets initiates counting. Travel time is minimum and the main significant differential comes from local processing. All counters must align with the master-electrode. The master-electrode and the slave-electrodes should count within a small differential. The small differential ensured that the slave-electrodes are synchronized with the master-electrode. Synchronization is obtained as long as the counting differential is smaller than the sampling interval of (1/300) second.

In some examples of the present invention, multiple electrodes may be self synchronized. In the alternate example, a master-electrode begins a transmission and awaits an acknowledgement from the slave-electrode. Upon receiving acknowledgement, a counter begins on all of the involved slave-electrodes. The master-electrode shall compare his clock (t=0) and received edges of slave's a crude time stamp is established. Assuming the transmission delay is negligible and the processing time is kept to a minimum such as 10⁻⁶ sec. The master electrode retransmits until all slave-electrodes acknowledge then and only then the synchronization process begins. In the case of a large delay i.e., more than a sampling time is observed, each unit knows of other unit clocks especially the master. Counters of the respective modules being to count up or down until the delay spread is minimized. The initial transmission is similar to wake up call to begin counting and to associate each counting with the number of sample or the time of the sample. The counting begins at the time of the master transmitting. Each macro-electrode begins to acknowledge each other's timing and predicts the current local sample number with respect to the virtual clock. Each slave-electrode reports its content to the master-electrode and each macro-electrode acknowledges its delay or equivalently early/late epochs. Each macro-electrode will adjust accordingly as they receive other clicks.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A macro-electrode device for remote sensing of a biophysiological signal comprising;
a substrate, said substrate comprising a plurality of sub-electrodes, said substrate forming one end of said macro-electrode;
a power source, said power source is removably coupled to said substrate; and,
a processing unit, said processing unit removably coupled to said power source;
wherein said substrate, power source and processing unit form an integrated, unitary device.

2. The macro-electrode of claim 1, wherein at least one sub-electrode is a receiver and at least one sub-electrode is an explorer.

3. The macro-electrode of claim 2, wherein at least one sub-electrode is a ground sub-electrode.

4. The macro-electrode of claim 1, wherein each sub-electrode is connected to the power source.

5. The macro-electrode of claim 1, wherein the power source is a battery.

6. The macro-electrode of claim 6, wherein the battery is rechargeable.

7. The macro-electrode of claim 1, wherein the power source has a power connection to the processing unit and a data transfer connection from each sub-electrode to the processing unit.

8. The macro-electrode of claim 1, wherein the processing unit comprises a means for acquiring data, a means for optimizing the biophysiological signal, a means for detecting an anomaly in the biophysiological signal, a means for transmitting the biophysiological signal, a means for storing data.

9. The macro-electrode of claim 8, wherein the processing unit further comprises means for transmitting and receiving speech.

10. The macro-electrode of claim 8, wherein at least two macro-electrode acquire a biophysiological signal and one macro-electrode is the master-electrode and the remaining macro-electrodes are slave-electrodes.

11. The macro-electrode of claim 10, wherein the biophysiological signals acquired are synchronized and the slave-electrode transmits data to the master-electrode and the master-electrode transmits the synchronized signal.

12. The macro-electrode of claim 1, wherein the biophysiological source is selected from the group consisting of skeletal muscle tissue, brain tissue, the eye, neurological tissue, nerve tissue, heart muscle, exposed brain tissue and epithelium tissue.

13. The macro-electrode of claim 1, wherein the substrate comprises a circuit board containing an amplifier.

14. A method of remote sensing of a biophysiological signal with a macro-electrode comprising the steps of:
acquiring the biophysiological signal;
filtering the biophysiological signal;
selecting the permutation of sub-electrodes that optimizes the filtered biophysiological signal wherein the optimized signal results in a baseline signal; and,
wirelessly transmitting the baseline signal to a receiver.

15. The method of claim 14, wherein the biophysiological signal is acquired from the group consisting of skeletal muscle tissue, brain tissue, the eye, neurological tissue, nerve tissue, heart muscle, exposed brain tissue and epithelium tissue.

16. The method of claim 14, wherein the biophysiological signal is acquired at a rate of per 1/300 second.

17. The method of claim 14, wherein the biophysiological signal is acquired between 0.5 Hz and 10,000 Hz.

18. The method of claim 14, wherein the biophysiological signal is filtered to obtain a signal between 0.5 Hz to 10,000 Hz.

19. The method of claim 14, wherein the biophysiological signal is filtered to obtain a signal between 0.5 Hz to 60 Hz.

20. The method of claim 14, wherein the biophysiological signal is filtered to obtain a signal between 0.5 Hz to 50 Hz.

21. The method of claim 14, wherein the optimizing the biophysiological signal is achieved by minimizing the noise and maximizing the signal.

22. The method of claim 14, wherein anomalies in the baseline signal are detected by interpreting the deviations from the pattern created by the baseline signal.
